(19) United States
(12) Patent Application Publication
(10) Pub. No.: US 2006/0004454 A1
(43) Pub. Date: Jan. 5, 2006

(54) Assembled disc spacers

(76) Inventors: Bret A. Ferree, Cincinnati, OH (US); David Tompkins, Milford, OH (US)

Correspondence Address: John G. Posa;Gifford, Krass, Groh, Sprinkle, Anderson & Citkowski, P.C., PO Box 7021, Troy, MI, 48007, US

(21) Appl. No.: 11/148,071

(22) Filed: Jun. 8, 2005

Related U.S. Application Data
(63) Continuation-in-part of application No. 10/422,282, filed on Apr. 24, 2003.
(60) Provisional application No. 60/375,212, filed on Apr. 24, 2002.

(51) Int. Cl. A61F 2/44

(52) U.S. Cl. 623/17.15; 623/17.11

(57) ABSTRACT

Disc spacers (DS) are assembled within a disc space, allowing them to be inserted through smaller openings in the Annulus Fibrosus (AF). The large size of the assembled DS decreases the pressure on the vertebral endplates. Embodiments of the invention may be used in the cervical, thoracic, or lumbar spine. The DS may be inserted through an anterior, lateral, or posterior approach to the spine using annulus preserving techniques. The devices are preferably made of biocompatible materials such as titanium, chrome cobalt, and ceramic. Alternative materials, including materials with shape memory properties, such as Nitinol, may be used to form one or more components of the device.

### REFERENCE TO RELATED APPLICATION

This application is a continuation-in-part of U.S. patent application Ser. No. 10/422,282, filed Apr. 24, 2003, which claims priority from U.S. Provisional Patent Application Ser. No. 60/375,212, filed Apr. 24, 2002, the entire content of both of which are incorporated herein by reference.

### FIELD OF THE INVENTION

This invention relates generally spine surgery and, in particular, to assembled disc spacers.

### BACKGROUND OF THE INVENTION

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Existing Artificial Disc Replacements (ADRs), also known as Total Disc Replacements (TDRs), have rigid endplates that are attached to the vertebra above and below the TDR. The large size of TDRs and the rigidity of TDRs make insertion from a posterior approach to the spine dangerous. My co-pending U.S. patent application Ser. No. 10/422,282, entitled “Artificial Intervertebral Disc Spacers,” teaches devices that articulate with the vertebral endplates and contain at least one joint within the device. The disc spacer devices may be inserted through a posterior approach to the spine.

### SUMMARY OF THE INVENTION

The present invention improves upon disc spacers (DS) in several ways. Importantly, the invention may be used to assemble DS within a disc space, allowing them to be inserted through smaller openings in the Annulus Fibrosus (AF). The large size of the assembled DS decreases the pressure on the vertebral endplates.

Embodiments of the invention may be used in the cervical, thoracic, or lumbar spine. The DS may be inserted through an anterior, lateral, or posterior approach to the spine. The DS are preferably inserted using the annulus preserving methods taught in my U.S. Pat. No. 6,878,167, the entire content of which is incorporated herein by reference.

The devices are preferably made of biocompatible materials such as titanium, chrome cobalt, and ceramic. Alternative materials, including materials with shape memory properties, such as Nitinol, may be used to form one or more components of the device.

### BRIEF DESCRIPTION OF THE DRAWINGS

FIG. 1A is a view of the top of the articulating side of a preferred embodiment of the present invention;

FIG. 1B is an exploded view of the top of the embodiment of the invention drawn in FIG. 1A;

FIG. 1C is a lateral view of the embodiment of the invention drawn in FIG. 1A;

FIG. 1D is an exploded lateral view of the embodiment of the invention drawn in FIG. 1B;

FIG. 1E is a lateral view of an assembled disc spacer in the preferred embodiment of the invention;

FIG. 1F is a coronal cross section of the spine and the embodiment of the invention drawn in FIG. 1E;

FIG. 2A is an axial cross section of a disc and one of the components of the embodiment of the invention drawn in FIG. 1A;

FIG. 2B is an axial cross section of the disc and the component of the DS drawn in FIG. 2A;

FIG. 2C is an axial cross section of the disc and two components of the embodiment of the invention drawn in FIG. 1A;

FIG. 2D is an axial cross section of the disc and the first two components of one half of a DS;

FIG. 2E is an axial cross section of the disc and the three components of the embodiment of the invention drawn in FIG. 1A;

FIG. 2F is an axial cross section of the disc and the embodiment of the invention drawn in FIG. 1A;

FIG. 3A is a coronal cross section of the spine and two components of the embodiment of the DS drawn in FIG. 1E;

FIG. 3B is a coronal cross section of the spine and four of the components of the embodiment of the invention drawn in FIG. 1E;

FIG. 3C is a coronal cross section of the spine and all six components of the embodiment of the invention drawn in FIG. 1E;

FIG. 4A is an axial cross section of one half of the DS drawn in FIG. 3C;

FIG. 4B is an axial cross section of one half of an alternative embodiment of the invention drawn in FIG. 4A;

FIG. 4C is an axial cross section of one half of an alternative embodiment of the invention drawn in FIG. 4B;

FIG. 5A is an axial cross section of an alternative embodiment of the invention drawn in FIG. 1A;

FIG. 5B is a coronal cross section of the embodiment of the invention drawn in FIG. 5A;

FIG. 5C is lateral view of the embodiment of the invention drawn in FIG. 5A;

FIG. 6 is a view of the articulating side of an alternative embodiment of the invention drawn in FIG. 1A;

FIG. 7A is a view of the articulating side of an alternative embodiment of the invention drawn in FIG. 1A;

FIG. 7B is an exploded view of the embodiment of the invention drawn in FIG. 7A;

FIG. 7C is a coronal cross section through the embodiment of the invention drawn in FIG. 7B;

FIG. 8 is a lateral view of an alternative embodiment of the invention drawn in FIG. 3A;

FIG. 9A is an exploded lateral view of an alternative embodiment of the invention drawn in FIG. 1E;

FIG. 9B is a view of the top of the embodiment of the invention drawn in FIG. 9A;

FIG. 10A is a view of the top of an alternative embodiment of the invention drawn in FIG. 1A;

FIG. 10B is an exploded view of the top of the embodiment of the invention drawn in FIG. 10A;

FIG. 10C is an axial cross section of the disc and the embodiment of the invention drawn in FIG. 10A;

FIG. 10D is a lateral view of the embodiment of the invention drawn in FIG. 10A;

FIG. 10E is an exploded view of the top of an alternative embodiment of the invention drawn in FIG. 10A;

FIG. 11A is a view of the top of an alternative embodiment of the invention drawn in FIG. 10A;

FIG. 11B is an exploded view of the top of the embodiment of the invention drawn in FIG. 11A;

FIG. 11C is a sagittal cross section through the embodiment of the invention drawn in FIG. 11A;

FIG. 11D is an axial cross section of the disc and the embodiment of the invention drawn in FIG. 11A; and

FIG. 11E is an axial cross section of the disc and the embodiment of the invention drawn in FIG. 11A.

### DETAILED DESCRIPTION OF THE INVENTION

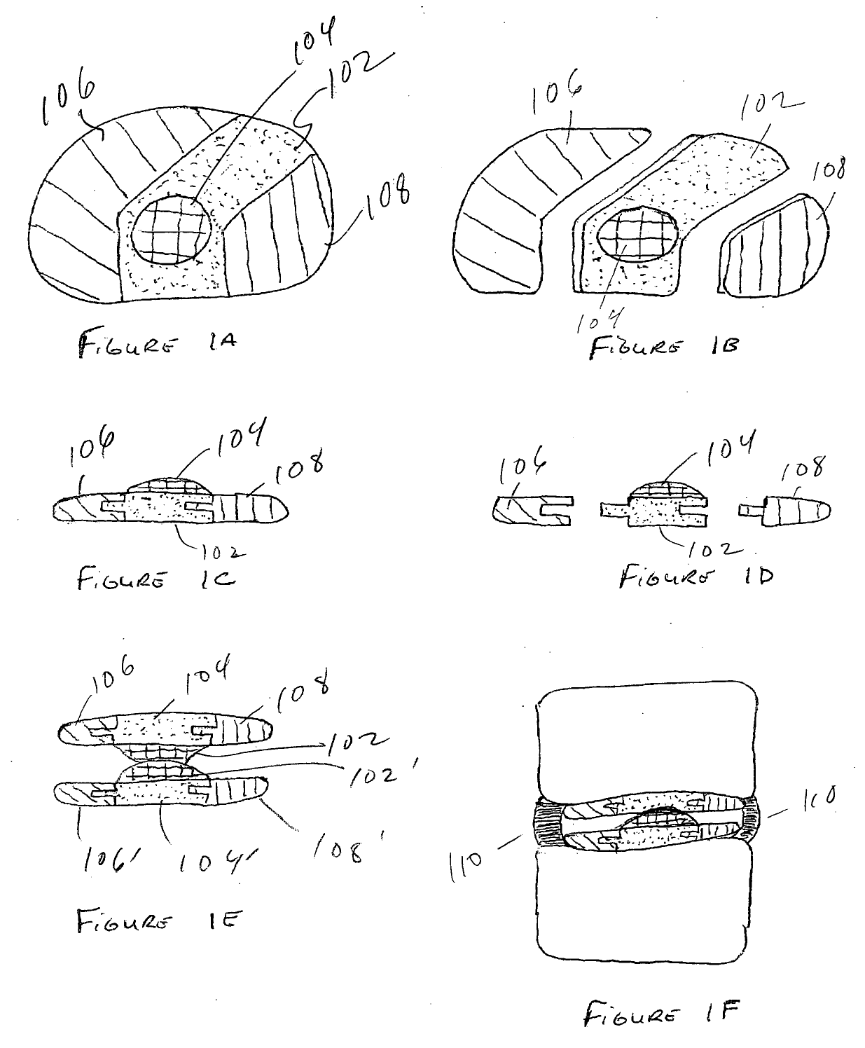

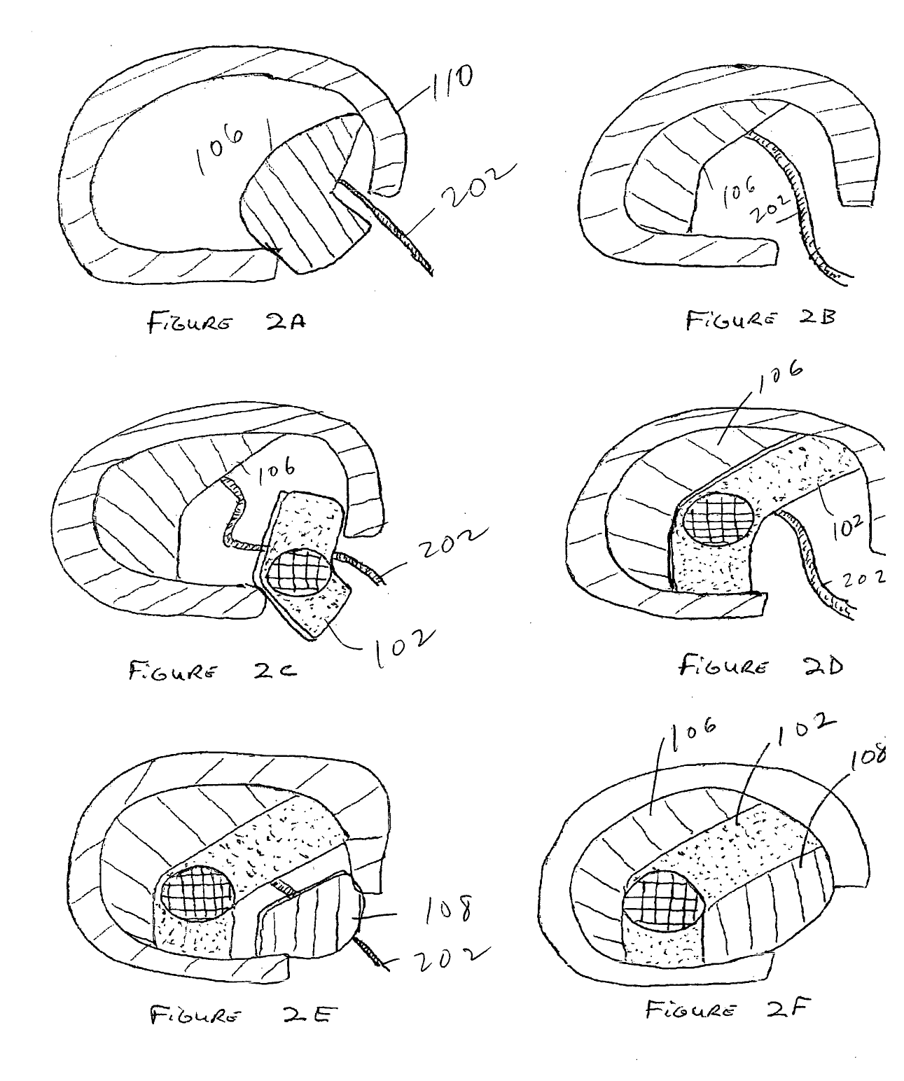

FIG. 1A is a view of the top of the articulating side of preferred embodiment of the invention. The assembled component may represent the top or bottom half of a disc spacer. The center component 102 contains a spherical articulating surface 104. The spherical articulating surface may be concave, convex, flat, or some combination thereof depending upon the opposing articulating surface. Separate components 106, 108 are located on either side of the component with the articulating surface.

FIG. 1B is an exploded view of the top of the embodiment of the invention drawn in FIG. 1A. FIG. 1C is a lateral view of the embodiment of the invention drawn in FIG. 1A. FIG. 1D is an exploded lateral view of the embodiment of the invention drawn in FIG. 1B. FIG. 1E is a lateral view of an assembled disc spacer in the preferred embodiment of the invention. The device is made of six components.

FIG. 1F is a coronal cross section of the spine and the embodiment of the invention drawn in FIG. 1E. The DS articulates with the vertebral endplates (VEPs). The top half of the device also articulates with the bottom half of the device. The Annulus Fibrosus (AF) of the disc is shown at 110.

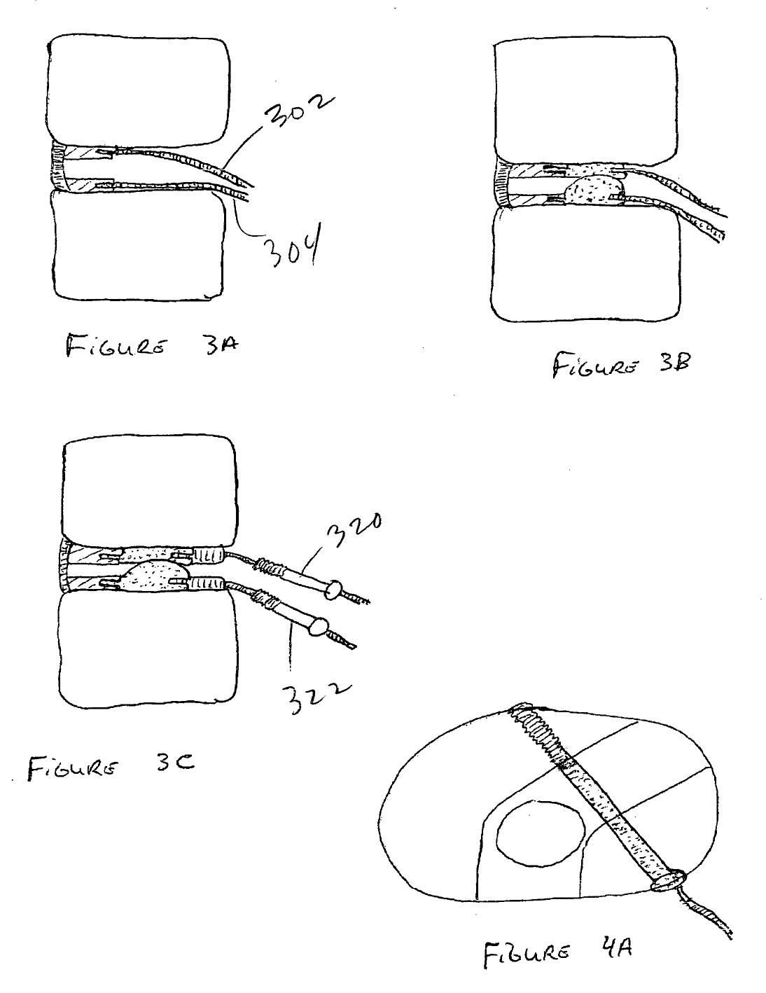

FIG. 2A is an axial cross section of a disc and one of the components of the embodiment of the invention drawn in FIG. 1A. The AF is again shown at 110. A wire, cable, band or cord 202 extends from the DS component. The DS component has been inserted into an opening in the AF. FIG. 2B is an axial cross section of the disc and the component of the DS drawn in FIG. 2A. The first DS component is positioned against the inner aspect of the AF on the side of the disc opposite the opening into the disc. The DS component may be rotated into position.

FIG. 2C is an axial cross section of the disc and two components of the embodiment of the invention drawn in FIG. 1A. The second component is threaded over the cable that extends from the first component. FIG. 2D is an axial cross section of the disc and the first two components of one half of a DS. The tongue-like projection from the side of the second component fits into a groove in the side of the first component.

FIG. 2E is an axial cross section of the disc and the three components of the embodiment of the invention drawn in FIG. 1A. The third component has been threaded over the cable that extends from the first component. FIG. 2F is an axial cross section of the disc and the embodiment of the invention drawn in FIG. 1A. The large DS is assembled within the disc space. The cable guides second and third components to the first component. The invention enables insertion of each component through a small opening in the AF. Each DS component may be rotated independent of the other components during the insertion process. Tongue and groove connections between the components enable each component to transmit axial loads to the adjacent components. The angle formed between the components helps prevent the components from sliding in an anterior to posterior direction relative to one another. Two or more cables may extend from the first component. The use of two or more cables facilitates alignment of the components within the disc space. Crimps may be placed over the ends of the cables to help hold the components together. Fluoroscopy or other navigational system may be used to help guide assembly of the DS within the disc space.

FIG. 3A is a coronal cross section of the spine and two components of the embodiment of the DS drawn in FIG. 1E. The first components of the top and the bottom half of the DS have been inserted into the disc space. Cables 302, 304 extend from each component. The AF is represented by the area of the drawing with horizontal lines. FIG. 3B is a coronal cross section of the spine and four of the components of the embodiment of the invention drawn in FIG. 1E. The components with the articulating surfaces have been threaded over the cables that extend from the first components of the top and the bottom half of the DS. The top and bottom articulating components are preferably inserted simultaneously into the disc with the convex spherical projection from one component contained within the spherical recess in the second component. The disc space may be distracted to facilitate insert of the DS. For example, rods or screws may be placed in the pedicles of the vertebra above and below the disc. A tool may be used to separate the pairs of rods or screws, thus enlarging the disc space.

FIG. 3C is a coronal cross section of the spine and all six components of the embodiment of the invention drawn in FIG. 1E. The DS has been assembled within the disc space. Cannulated screws 320, 322 may be threaded over the cables to hold the assembled DS components together. One or more screws may be passed over one or more cables extending from each half of the assembled DS.

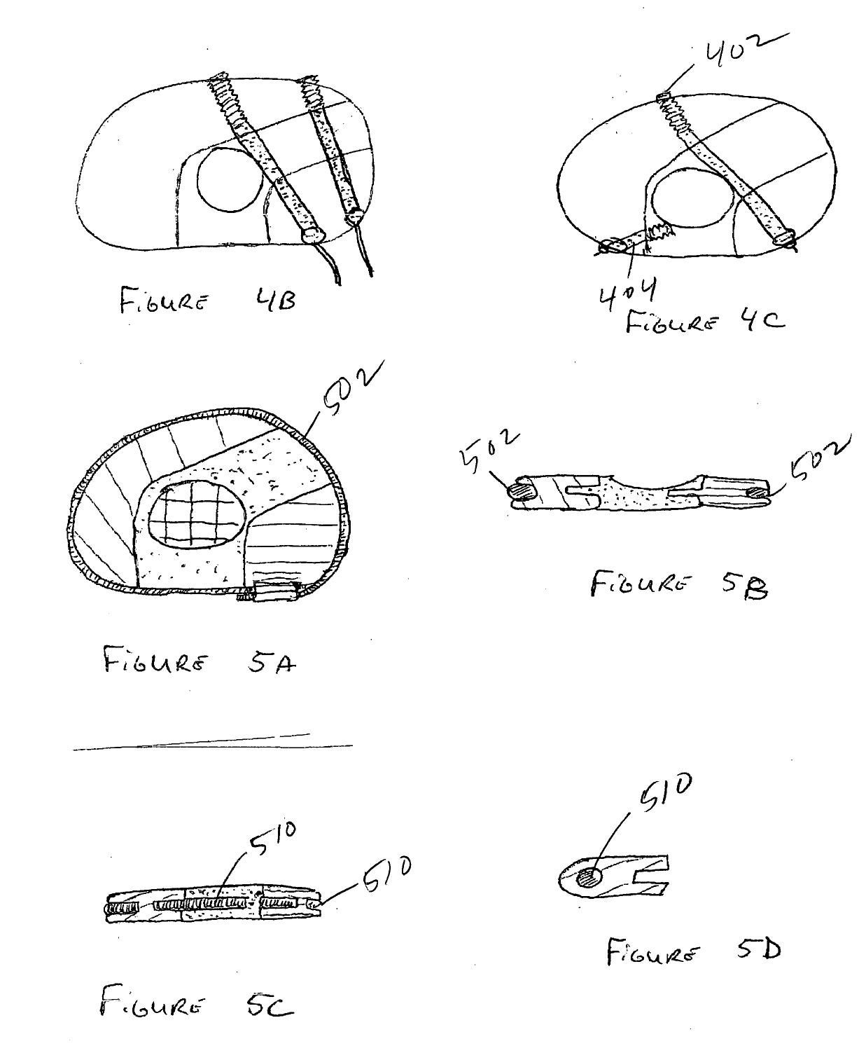

FIG. 4A is an axial cross section of one half of the DS drawn in FIG. 3C. The screw holds the DS components together. The cable may be cut after placement of the screw. Alternatively the cable may be crimped and cut after placement of the screw. More than one screw and/or cables may be used in each half of the assembled DS. For example, the preferred embodiment of the invention may use two cables and one screw per half of the DS. The screw may pass over one of the cables.

FIG. 4B is an axial cross section of one half of an alternative embodiment of the invention drawn in FIG. 4A. FIG. 4C is an axial cross section of one half of an alternative embodiment of the invention drawn in FIG. 4B. Screws 402, 404 have been inserted from the left and the right sides of the spine. Crimps have been placed over the ends of the cables. The cables have been cut.

FIG. 5A is an axial cross section of an alternative embodiment of the invention drawn in FIG. 1A. A cable 502 surrounds the three components of the top or bottom of the DS. The ends of the cable have been placed into to a crimp component (rectangle on the bottom right corner of the drawing). Cable ties could be tightened around the assembled top and bottom halves of the device in alternative embodiments of the invention. FIG. 5B is a coronal cross section of the embodiment of the invention drawn in FIG. 5A. The cable sits in a recess around the periphery of the device. Alignment cables similar to those drawn in FIG. 2E may be used in conjunction with this embodiment of the invention.

FIG. 5C is lateral view of the embodiment of the invention drawn in FIG. 5A. The cable 510 may be threaded into a recess surrounding each half of the DS. FIG. 5D is a partial coronal cross section of the embodiment of the invention drawn in FIG. 5C. The cable is partially enclosed within holes of the DS components.

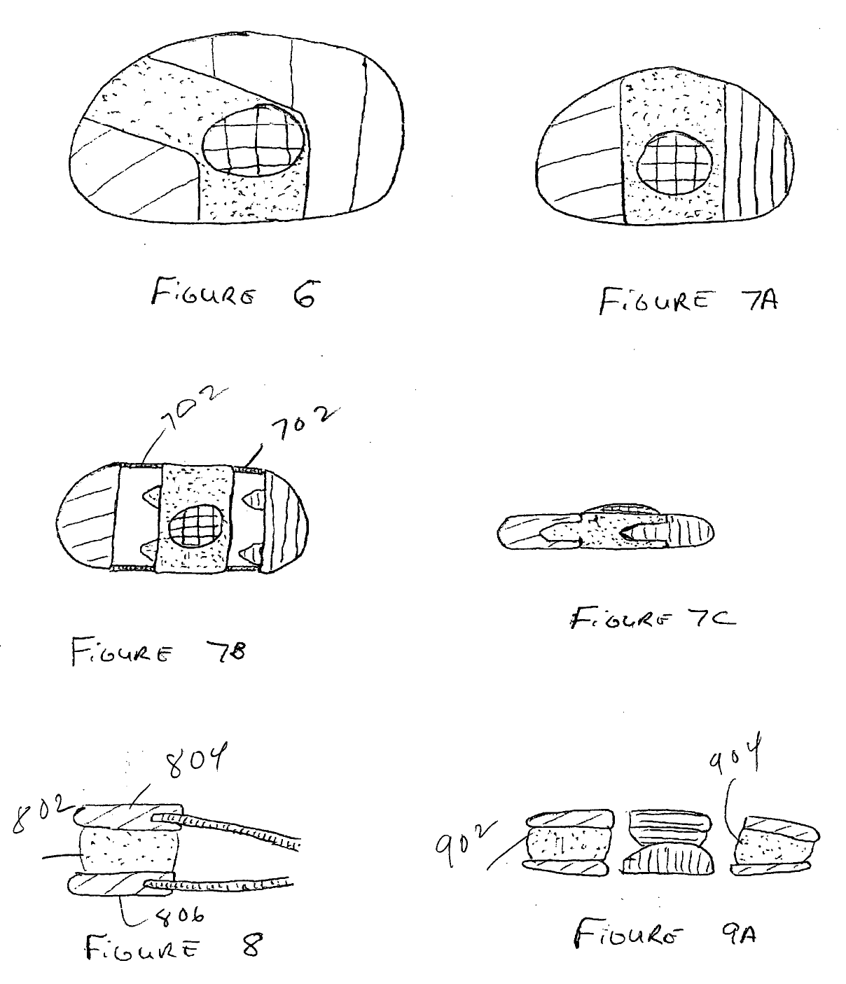

FIG. 6 is a view of the articulating side of an alternative embodiment of the invention which is essentially a mirror image of the embodiment of the invention drawn in FIG. 1A. The DS drawn in FIG. 1A was designed for insertion through an opening in the right posterior coroner of the disc. The embodiment of the DS drawn in FIG. 6 was designed for insertion through an opening in the left side of the disc.

FIG. 7A is a view of the articulating side of an alternative embodiment of the invention drawn in FIG. 1A. The drawing represents one half of a DS. Each half of the DS preferably has three components. FIG. 7B is an exploded view of the embodiment of the invention drawn in FIG. 7A. A cable 702 surrounds the periphery of the device. Cone projections from one component fit into conical holes in the adjacent component. The cable is tightened and fastened after the DS is assembled within the disc space. FIG. 7C is a coronal cross section through the embodiment of the invention drawn in FIG. 7B. The cross section was made through the cones.

FIG. 8 is a lateral view of an alternative embodiment of the invention wherein a bio-resorbable material 802 has been placed between the top and bottom components 804, 806 of the DS. The bio-resorbable material holds the two DS components in optimal alignment. The alignment of the first DS components facilitates assembly of the DS in the disc space. The first two DS components are held in a position that aligns the components with the articulating DS components. The spacer material resorbs to allow movement between the two halves of the DS after DS is inserted in the disc space. A material such as sterile ice or frozen saline would allow rapid melting and resorption of the component. Alternative materials could be used for slower resorption of the component. Bio-resorbable materials could include: polylactic acid (PLA), polyglycolic acid (PGA), poly (ortho esters), poly(glycolide-co-trimethylene carbonate), poly-L-lactide-co-6-caprolactone, polyanhydrides, poly-n-dioxanone, and poly(PHB-hydroxyvaleric acid.

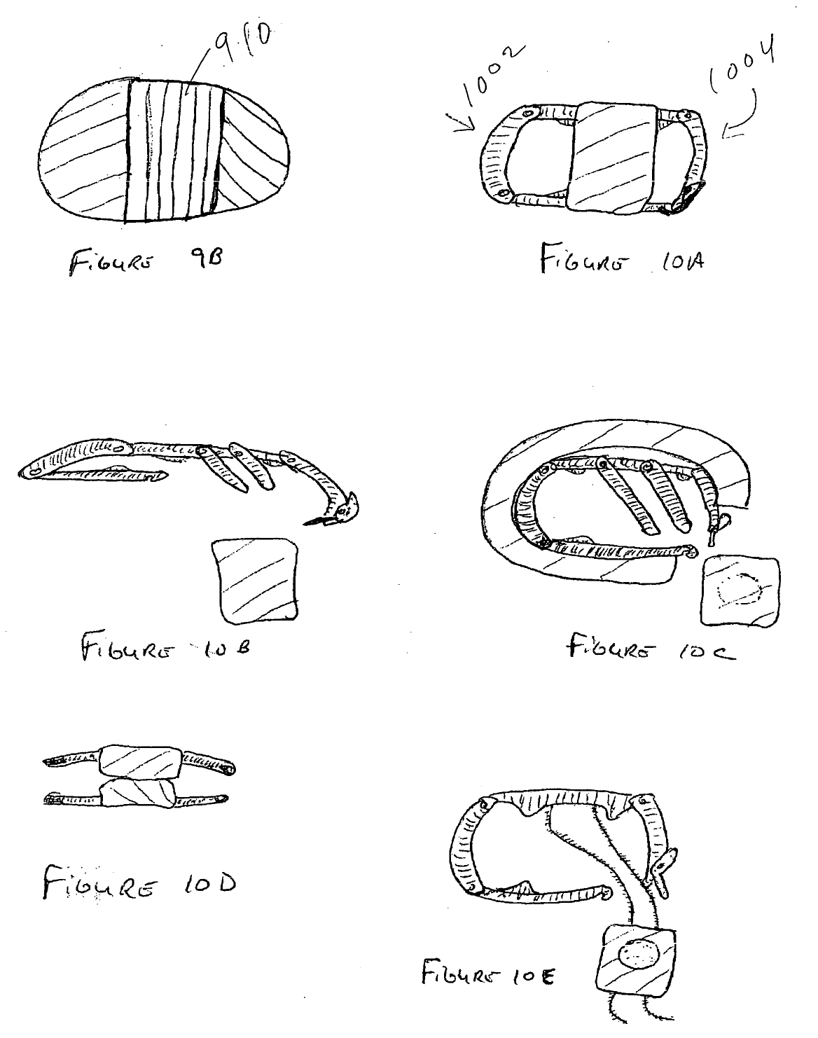

FIG. 9A is an exploded lateral view of an alternative embodiment of the invention wherein the center portions of the DS have spherical articulating surfaces. The lateral portions of the DS have compressible components 902, 904 between the top and bottom halves of the device. For example, an elastomer may be bonded to the top and or the bottom halves of the lateral portions of the device. Alternatively, a spring or springs could be placed between the top and bottom halves of the lateral DS components. The lateral DS components are not attached to the central, articulating, DS components in the preferred embodiment of the invention. FIG. 9B is a view of the top of an embodiment of the invention including a central, articulating portion 910.

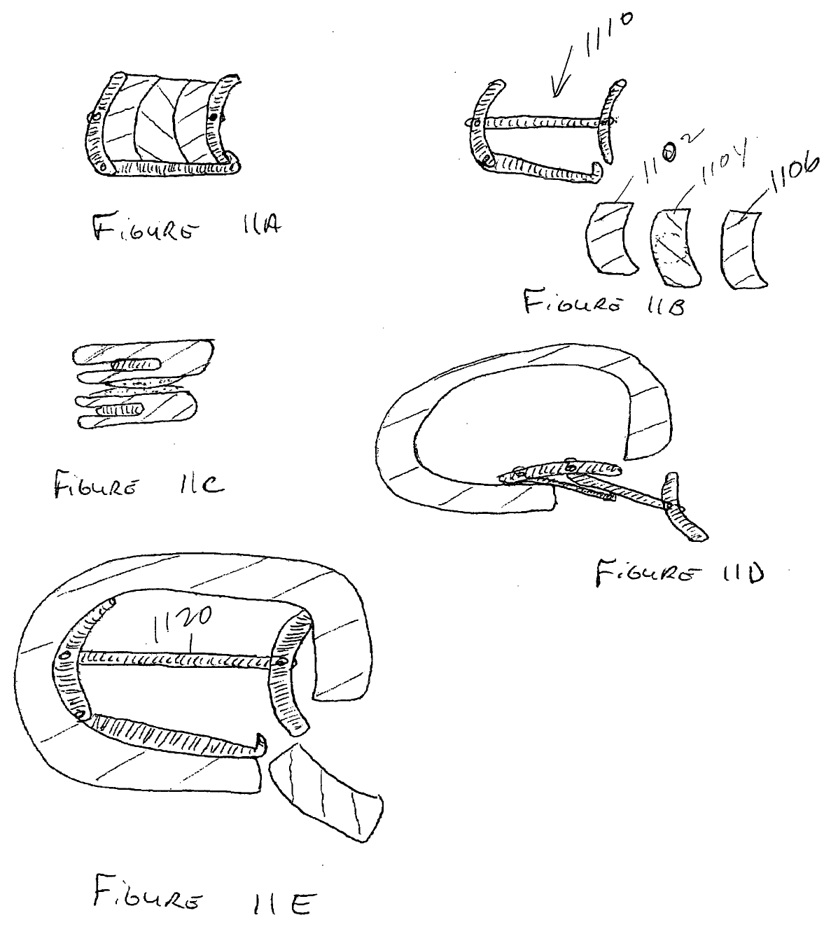

FIG. 10A is a view of the top of an alternative embodiment of the invention. FIG. 10B is an exploded view of the top of the embodiment of the invention drawn in FIG. 10A. Collapsible frames 1002, 1004 are inserted into the disc space. Articulating components are placed over extensions from the collapsible frame.

FIG. 10C is an axial cross section of the disc and the embodiment of the invention drawn in FIG. 10A. The frame has been expanded within the disc. The articulating component is seen outside the disc. The dotted circle represents the spherical articulating surface of the central component. The articulating components are placed over the vertical arms extending from the center of the collapsible frame. The ends of the vertical arms of the frame may fit into holes in the base of the frame. The frame has a claming mechanism to hold the frame closed after the articulating components are inserted onto the frame. The articulating components force the top and bottom frame components apart. The elasticity of the frame and the strap or clamp help to hold the frame in its closed configuration. FIG. 10D is a lateral view of the embodiment of the invention drawn in FIG. 10A. The DS has top and bottom halves.

FIG. 10E is an exploded view of the top of an alternative embodiment of the invention drawn in FIG. 10A. The articulating components are threaded over cables extending from the frame. The cables may be cut after the DS is assembled and the frame has been fastened.

FIG. 11A is a view of the top of an alternative embodiment of the invention. FIG. 11B is an exploded view of the top of the embodiment wherein three components 1102, 1104, 1106 are placed over a collapsible frame 1110. The center component has an articulating surface. The dotted circle represents the articulating surface. The assembled DS has a top and a bottom frame.

FIG. 11C is a sagittal cross section through the embodiment of the invention drawn in FIG. 11A. The cross section was taken through the articulating portion of the device. The cross bar is shown at 1120. The cross bars of the frames fit within slots spacer components. FIG. 11D is an axial cross section of the disc and the embodiment of the invention drawn in FIG. 11A. The frame has been collapsed to facilitate insertion through a small opening in the AF.

FIG. 11E is an axial cross section of the disc and the embodiment of the invention drawn in FIG. 11A. The first spacer component is seen just outside the disc. A locking component is used to hold the spacer components on the frame. For example, a strap could be attached across the bottom of the assembled device. Elastic properties in the frame and/or strap may facilitate the fastening mechanism.

We claim:

1. A disc spacer, comprising:
a plurality of components, including at least one component with an articulating surface; and
wherein the components are interlocking, facilitating assembly of the components within an intervertebral disc space.

2. The disc spacer of claim 1, wherein the components interlock using a tongue-and-groove structure.

3. The disc spacer of claim 1, including three components, and wherein the component with the articulating surface is central to the other two.

4. The disc spacer of claim 1, wherein the articulating surface is concave or convex.

5. The disc spacer of claim 1, wherein the components are held together with screws.

6. The disc spacer of claim 1, wherein the components are held together with an encircling band.

7. The disc spacer of claim 1, wherein the components are held together with an articulating frame.

8. The disc spacer of claim 1, wherein one of the components includes one or more cables or wires onto which a subsequent component may be journaled to coordinate the assembly thereof.

9. The disc spacer of claim 1, wherein the components are held together with an articulating frame.

10. The disc spacer of claim 1, including an upper and lower set of interlocking components, each set including a components with an articulating surface.

11. A method inserting a disc spacer, comprising the steps of:
providing the components of claim 1;
making an incision in an annulus fibrosis; and
sequentially inserting the components through the incision; and
assembling the components in situ.

12. The method of claim 11, wherein components are inserted using an anterior approach.

13. The method of claim 11, wherein components are inserted using a lateral approach.

14. The method of claim 11, wherein components are inserted using a posterior approach.
